(19) United States
(12) Patent Application Publication
(10) Pub. No.: US 2016/0302740 A1
(43) Pub. Date: Oct. 20, 2016

(54) COMPACT SCANNER ASSEMBLY WITH A RESONANT SCANNER AND TWO GALVANOMETER SCANNERS FOR MULTI REGION OF INTEREST (MROI) IMAGING AND TARGETING OF INTACT TISSUE

(71) Applicant: VIDRIO TECHNOLOGIES, LLC, Arlington, VA (US)

(72) Inventors: Vijay IYER, Arlington, VA (US); Nathan CLACK, Arlington, VA (US); Ed KANG, Arlington, VA (US); Georg JAINDL, Arlington, VA (US)

(21) Appl. No.: 15/196,407

(22) Filed: Jun. 29, 2016

Related U.S. Application Data
(60) Provisional application No. 62/142,955, filed on Apr. 3, 2015.

(51) Int. Cl. A61B 5/00; G02B 26/10; G02B 21/00; A61N 5/06; A61B 90/20

(52) U.S. Cl. CPC A61B 5/748; A61B 5/0042; A61N 5/0622; A61B 5/0062; A61B 90/20; G02B 21/006; G02B 21/0048; G02B 26/101; G02B 21/0012; A61B 2018/00577

(57) ABSTRACT

Provided herein are apparatuses and methods using a compact scanner assembly for multi region of interest (MROI) imaging and targeting of an intact tissue sample. The compact scanner assembly for MROI imaging, comprises a set of three electromagnetically actuated scanning mirrors, the first comprising a resonant scanner (R) driven at its resonant frequency, the second comprising a galvanometer scanner (G1) having a mirror (m1), the third comprising a galvanometer scanner (G2) having a mirror (m2), wherein the galvanometer scanners (G1) and (G2) are driven by a lower bandwidth control signal specifying an angle for mirror (m1) and (m2), wherein the scanners are arranged sequentially as (R) (G1) (G2), wherein the set of three scanning mirrors are within a single scanner assembly (RGG), wherein the compact scanner assembly for MROI combines raster scanning with random-access scanning to target multiple ROIs for targeting and imaging.

### STATEMENT REGARDING FEDERALLY SPONSORED RESEARCH OR DEVELOPMENT

This invention was developed and/or researched using federal government funds under NIH Contract #______.

### CROSS REFERENCE TO RELATED APPLICATIONS

Not applicable.

### NAMES OF PARTIES TO A JOINT RESEARCH AGREEMENT

Not applicable.

### BACKGROUND

1. Field of the Invention

This invention relates to a compact scanner assembly with a resonant scanner and two galvanometer scanners for multi region of interest (mroi) imaging and targeting of intact tissue.

2. Background of the Invention

Two-photon laser scanning microcopy (2PLSM) is a method for high-resolution, three-dimensional imaging in intact tissue. For the past 2 decades, 2PLSM has enabled celllar resolution in vivo brain imaging [ ]. Within the last decade, experimental approaches and molecular tools have advanced to allow robust observation of cellular-resolution brain activity in awake behaving animals, such as: navigation in virtual reality environments, active motion, controlled sensory input, complex behavioral tasks, and measurable animal learning [ . . . ].

The cerebral cortex is widely understood to be organized into columns of 300-600 μm in diameter¹⁻⁴. Information in the brain is distributed and transformed across cortical columns and areas (FIG. 1a). Such coordination between brain regions can be increasingly correlated to specific and measurable animal behavior, such as the coordination of contralateral cortical premotor regions in preparing a unilateral motion⁵.

Full understanding of information processing in the brain requires cellular resolution activity mapping of two or more interacting cortical columns during specific behaviors. But today's widely used 2PLSM optics and objectives reach fields-of-view (FOV) of only up to ˜500 μm diameter, i.e. corresponding to the size of a single cortical column. Newly reported wide-FOV scanning optics are more than doubling the scanned area with current-day objectives (to >1 mm diameter); and new wide-FOV objectives are expanding the area even further, reaching up to 3.5 mm diameter⁶. These field sizes span beyond individual and adjacent columns to cover much broader cortical regions—indeed nearly the entire visual cortex of the mouse brain.

These new optics provide an exciting avenue to allow large-scale optical recordings of neural activity in multiple cortical regions concurrently. But a key additional development is required to maintain both imaging speed and signal quality comparable to today's optical recordings, across such wider FOVs. Imaging must be targeted to specific cortical regions-of-interest (ROIs), in order to ensure that neither laser scanning time, nor the proportional signal integration time, is wasted by imaging across large intervening areas not under study.

Market Outlook and Commercialization

Certain software products are widely used for 2PLSM applications in neurobiology (FIG. 2), such as for example Vidrio Technologies ScanImage software product. Market research indicates there are at least 1000 2PLSM systems worldwide for cellular brain imaging, with approximately 100 new scope systems being sold each year. Thus, there is a great need for these products in the marketplace. Market interest is accelerating due to great advances in molecular indicators⁷ that are narrowing the gap in spike sensitivity and signal fidelity compared to electrical recordings. Numerous researchers previously focused exclusively on electrical recording approaches are switching to or adding optical recording techniques to their toolkit. The vast majority of deployed 2PLSM systems are galvo-galvo (GG) scanner systems. Because of the speed, signal and specimen quality, and motion tolerance benefits of resonant scanning^{8,9}, the majority of these users have a strong desire to benefit by upgrading to an RG raster scanning system.

### SUMMARY OF THE INVENTION

We provide the alternative of Multi-ROI (MROI) Imaging which combines raster scanning with random-access scanning, to target multiple geometric ROIs, e.g. rectangular, linear, 3-D spaces, for imaging (FIG. 1b). MROI imaging employs a combination of scanner components: one resonant scanner and two galvanometer (galvo) scanners, arranged sequentially within a single scanner assembly (RGG).

MROI imaging based on RGG scanner hardware can be retrofitted into existing 2PLSM systems to reach wide 1 mm FOV diameters, provided that a compact assembly with sufficient size and angular range is designed and produced (Aspect 1). Dedicated MROI imaging system software will coordinate targeted scanning in each plane with an axial sweep, allowing simultaneous optical recordings from neural ensembles (>100) in multiple volume ROIs at >4 Hz (Aspect 2; FIG. 1c).

Phase 2 work adapting MROI imaging to wider-FOV objectives requires a more optimized optical design⁶, forming a complementary hardware option to the compact design achieved in Phase 1. Adaptive and stepped axial scan modes are added in software to more precisely specify and shape the planes imaged during MROI volume imaging

MROI imaging allows scientists to observe interacting cortical regions in the mammalian brain at multiple spatial scales: cellular resolution imaging within distinct and separated anatomical and functional regions. Examples include the transformation of visual input from lower to higher-order representations and associations[ ] in primary visual cortex (V1) and the sensorimotor loop interactions[ ] between somatosensory (S1) and motor (M1) cortices (FIG. 1c).

### Preferred Embodiments

Accordingly, provided herein as one preferred embodiment is an apparatus for targeting a sample, the apparatus comprising: a source of beamed electromagnetic energy producing a beam; a compact scanner assembly for multi-region of interest (MROI) angular scanning of the beam; and optics for imaging the angular scanned beam from scanner assembly to a microscope objective lens, wherein the microscope objective lens converts the angular scanned beam to a focused spatial focusing scanned beam onto intact cellular tissue.

In another preferred embodiment is an apparatus for imaging a sample, the apparatus comprising: a source of beamed electromagnetic energy producing a beam; a compact scanner assembly for multi-region of interest (MROI) angular scanning of the beam; and optics for imaging the angular scanned beam from scanner assembly to a microscope objective lens, wherein the microscope objective lens converts the angular scanned beam to a focused spatial focusing scanned beam onto intact cellular tissue; and a detector to detect the resulting radiation signal from the sample region.

In another preferred embodiment is provided a compact scanner assembly for multi-region of interest (MROI) imaging of intact cellular tissue, comprising a set of three electromagnetically actuated scanning mirrors, the first scanning mirror comprising a resonant scanner (R) driven at its resonant frequency, the second scanning mirror comprising a galvanometer scanner (G1) having a mirror (m1), the third scanning mirror comprising a galvanometer scanner (G2) having a mirror (m2), wherein the galvanometer scanner (G1) and the galvanometer scanner (G2) are driven by a lower bandwidth control signal specifying an angle for mirror (m1) and for mirror (m2), wherein the scanners are arranged sequentially as the resonant scanner (R) followed by the galvanometer scanner (G1) and the galvanometer scanner (G2), wherein the set of three scanning mirrors are within a single scanner assembly (RGG).

In another preferred embodiment is provided the compact scanner assembly, wherein the set of three scanning mirrors are oriented within the compact scanner assembly to produce a two-dimensional (X & Y) angular scan at an assembly output, wherein the resonant scanner (R) and the galvanometer scanner (G1) are oriented to produce scanning in the same (X) angular direction.

In another preferred embodiment is provided the compact scanner assembly, wherein the compact scanner assembly for MROI combines raster scanning with random-access scanning to target multiple geometric ROIs for imaging.

In another preferred embodiment is provided the compact scanner assembly, wherein the geometric ROIs comprise a linear ROI, a planar ROI, or a three-dimensional ROI.

In another preferred embodiment is provided the compact scanner assembly, wherein the geometric ROIs comprise a rectangular ROI, a line ROI, a curved line ROI, a circular ROI, an elliptical ROI, a multi-sided ROI, a polygon ROI, an irregular ROI, a cubic ROI, a prismatic ROI, a cylindrical ROI, or a spherical ROI.

In another preferred embodiment is provided a method of imaging a sample, comprising: scanning a beam of electromagnetic energy over an intact tissue sample; using a compact scanner assembly for multi-region of interest (MROI) imaging of intact cellular tissue disposed to receive the beam; and detecting the resulting radiation signal from the sample region.

In another preferred embodiment is provided a method for providing multi region cellular resolution imaging, comprising the steps: providing a device as herein, using the device to obtain MROI images.

In another preferred embodiment is provided a method of targeting a sample of cellular tissue, comprising: directing a beam of electromagnetic energy at an intact tissue sample; and using a compact scanner assembly for multi-region of interest (MROI) targeting of intact cellular tissue disposed to receive the beam.

In another preferred embodiment is provided a method for providing multi region cellular resolution targeting, comprising the steps: providing a device as herein, using the device to target cellular tissue.

In another preferred embodiment is provided the method as herein, wherein the cellular tissue is a synapse.

In another preferred embodiment is provided the method as herein, further comprising the step of ablating of the targeted cells.

In another preferred embodiment is provided the method as herein, further comprising the step of optogenically stimulating the targeted cells.

In another preferred embodiment is provided the method as herein, further comprising the step of photo-stimulating the targeted cells.

In another preferred embodiment is provided the methods as herein, wherein the compact scanner assembly for MROI combines raster scanning with random-access scanning to target multiple geometric ROIs for targeting wherein the geometric ROIs comprise a linear ROI, a planar ROI, or a three-dimensional ROI.

In another preferred embodiment is provided the method as herein, wherein the geometric ROIs comprise a rectangular ROI, a line ROI, a curved line ROI, a circular ROI, an elliptical ROI, a multi-sided ROI, a polygon ROI, an irregular ROI, a cubic ROI, a prismatic ROI, a cylindrical ROI, or a spherical ROI.

### BRIEF DESCRIPTION OF THE FIGURES

FIG. 1 is a three part (a), (b), (c) graphic. FIG. 1a show information flows between cortical columns. FIG. 1b shows MROI imaging combines random access and raster scanning. FIG. 1c shows example applications of MROI optical recordings.

FIG. 2 is a bra graph. FIG. 2 shows growing needs for products in the marketplace that provide solutions offered by the present invention.

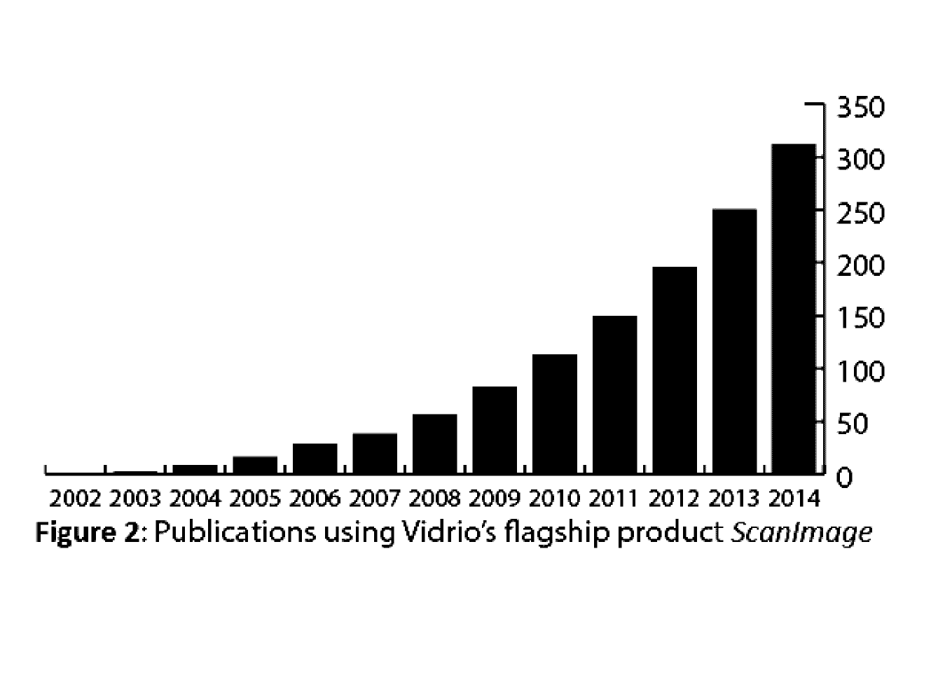

FIG. 3 is a two part graphic, (a) and (b). FIG. 3a shows RGG scanner combination for MROI imaging. FIG. 3b shows scanner characterization by diameter and scan angle.

FIG. 4 is an image. FIG. 4 shows volume imaging and MROI.

FIG. 5 is a graphic illustration. FIG. 5 shows a compact scanner assembly for MROI imaging.

FIG. 6 is a graphic illustration. FIG. 6 shows MROI volume imaging graphed against axial sweep.

### DETAILED DESCRIPTION OF THE INVENTION

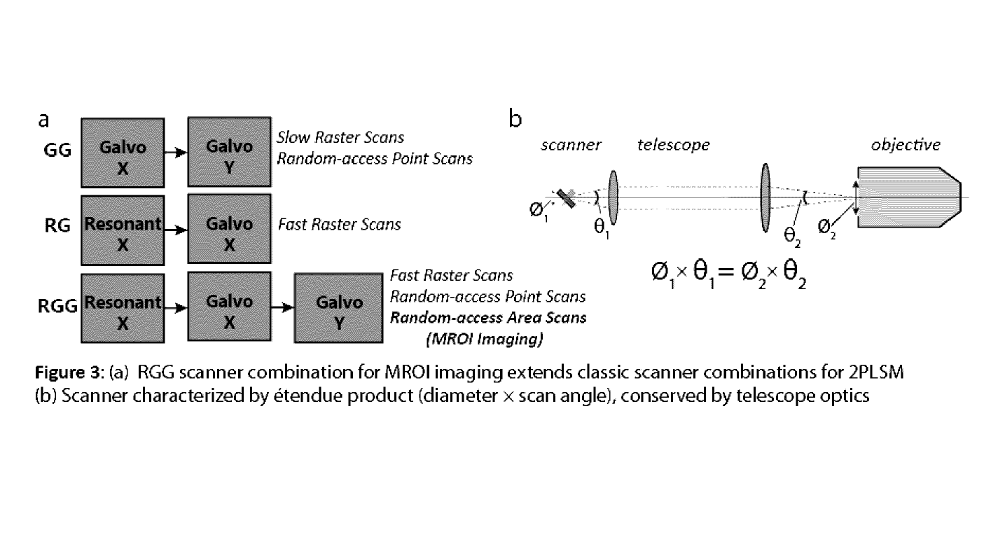

Referring now to FIG. 3, Multi Region-Of-Interest (MROI) imaging is a resonant-galvo-galvo (RGG) scanning approach that extends widely used RG raster scanning and GG random-access scanning approaches (FIG. 3a) for in vivo 2PLSM to allow two innovative new capabilities: random-access imaging, rather than point or contour random-access scanning¹⁰⁻¹², to allow robustly motion correctable optical recordings across a wide FOV multi-region cellular resolution imaging with greater compatibility to existing microscopes and greater scalability, to beyond 2 regions, than existing approaches^{6,13} RGG scanning may also be used in other key applications in brain science.

Random-Access Imaging

Previous random-access laser scanning approaches described to date have employed galvo or acousto-optic deflector^{12,14-17} scanning pairs. Galvo scan bandwidths are limited to ˜1 kHz line rates. For this reason, random-access optical recordings with GG scanner pairs have employed complex scan trajectory based on parametric^{10,18} or heuristic¹¹ optimization of scanner trajectory to maximize the recording rate and the number of sites recorded. However, such approaches are only suited for motion-free in vitro applications; locking such complex scans during in vivo imaging would require highly complex registration and control heuristics.

Acousto-optic deflectors (AODs) allow extremely fast (inertia-free) scans suited for multi-area scanning, but are not currently suited for use with wide-FOV optics due to their limited scanner étendue¹⁹, defined as the product of the optical aperture and angular range (FIG. 3b). Étendue is an invariant quantity preserved by any subsequent optics imaging the scanner to the objective back entrance aperture. Current wide FOV objectives (Nikon CFI 75 16×) have back entrance apertures ˜20 mm and entrance angular ranges of +/−4°. Filling the aperture (to achieve full resolution) and angular range (to reach full extent of the FOV) would require a scanner étendue of 160 mm-degrees; with even larger values required for emergent wider FOV objectives⁶. AODs suited for 2PLSM are currently limited to only <50 mm-degrees and thus cannot fully utilize even today's wide-FOV objectives¹⁵.

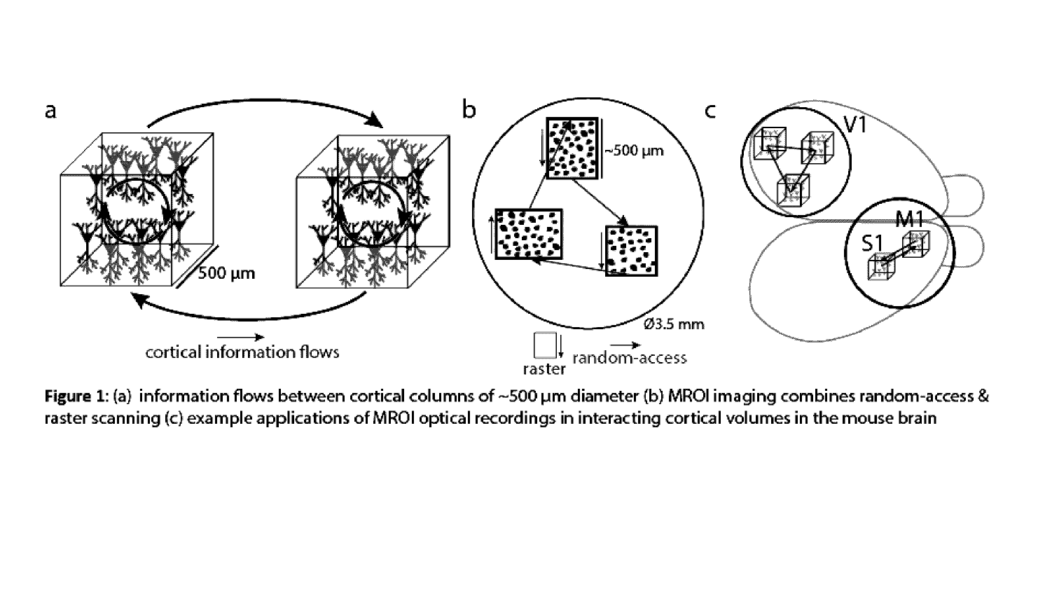

Referring now to FIG. 1, The Multi-ROI Imaging strategy overcomes these limitations by combining the 16 kHz line rate of resonant scanning (Cambridge Technology CRS 8) with 2D random-access GG scanning to allow random-access imaging (FIG. 1b). Galvo scanners used for 2PLSM can reach large étendue values of 240 mm-degrees (e.g. the Cambridge Technology 6215 6 mm, +/−20° scanner). Moreover, their limited scan speed does not greatly limit MROI imaging: the GG transit time (100-500 μs) is negligible, to first order, between each rectangular ROI in comparison to each ROI imaging period. The photon efficiency of targeting the laser scan only to ROIs can be flexibly allocated to achieve gains in imaging speed, pixel integration time, and/or image resolution, compared to the reference full FOV raster scan (Table 1).

TABLE 1
Table 1: Example Multi-ROI Imaging scenarios

| | | | Frame | | |
|---|---|---|---|---|---|
| ROI | | # | Rate | (Pixel Time | |
| Zoom | Pixelation | ROIs | (Hz) | (normalized) | Comments |
| 1 | 256 × 256 | 1 | 60 | 1 | Reference RG |
| | | | | | raster of full |
| | | | | | FOV |
| 4 | 64 × 64 | 2 | 120 | 4 | Max gain in |
| | | | | | frame speed |
| 4 | 64 × 64 | 3 | 80 | 4 | |
| 4 | 64 × 64 | 4 | 60 | 4 | Pure gain in ROI |
| | | | | | signal quality |
| 4 | 128 × 128 | 2 | 60 | 2 | Gain in ROI line |
| | | | | | resolution |

Multi-Region Imaging

Two very recent reports have described new technologies to address the challenge identified by this proposal: simultaneous cellular-resolution imaging of multiple separated cortical regions.

One technology circumvents the limited FOV of standard microscope systems by using two independent microendoscopic paths¹³. Another technology, Trepan2P, employs two parallel GG scanner pathways, together with temporal multiplexing, to achieve targeted two-area imaging⁶.

Compared to MROI imaging, both of these techniques are considerably more complex to implement and align; moreover, neither readily scales to beyond two regions. Moreover Trepan2P is not readily extensible to 10× faster resonant (RG) raster imaging, so that its frame rates are slower than MROI imaging (Table 1) despite truly simultaneous dual region imaging.

MROI imaging hardware is, in contrast, readily inserted as a plug-and-play upgrade to existing 2PLSM systems, reaching at least >1 mm diameters as provided here (Aspect 1).

Other RGG Scanning Applications

Notably, the same core RGG-scanning technology, combining random and raster-access scanning, can be applied to other key applications in brain science. For instance, MROI imaging achieves fast, photon-efficient imaging for subcellular scale applications, such as comprehensive mapping of synaptic input to a single neuron. RGG scanning may also be applied to multi-site photostimulation applications using optogenetics²⁰, which has been recently made compatible with two-photon excitation using area scanning approaches targeted to neural cell bodies^{21,22}. Finally, RGG scanning is applicable to neural imaging and photostimulation applications in other brain areas [ ] and other model organisms such as the fly [ ] and zebrafish [ ] that are accessible by 2PLSM. These uses broaden the impact of this research.

Approach

Preliminary Data

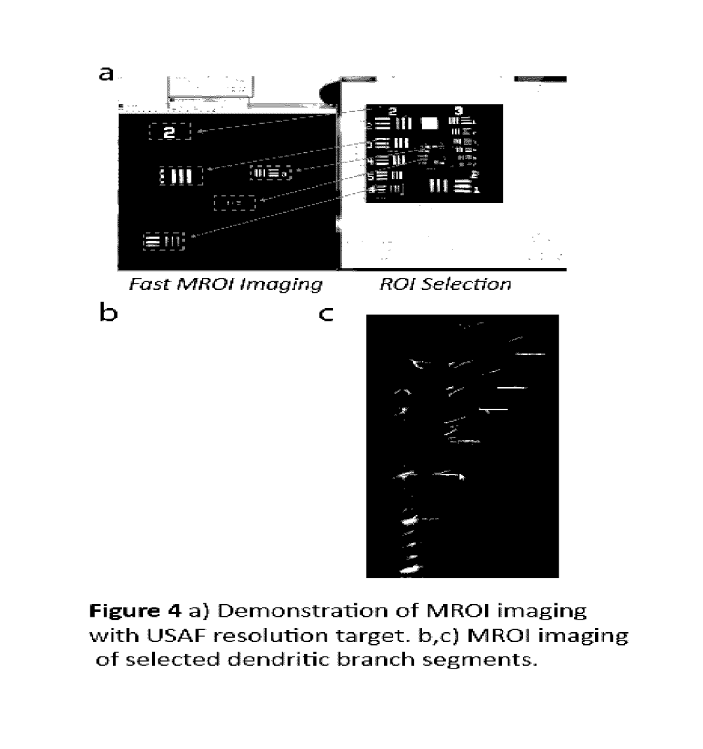

Referring now to FIG. 4, Vidrio Technologies flagship ScanImage product has been widely used for single cortical region (column) in vivo imaging applications in the mammalian brain [ . . . ], including recent applications using resonant scanned volume imaging [ . . . ] (FIG. 4a). One of Vidrio Technologies' customers (HHMI/Janelia Research Campus) has constructed a custom optical system that includes a resonant scanner and galvo scanner pair in sequence. Vidrio has developed alpha-level software for this customer demonstrating the concept of MROI imaging (FIG. 4b). Recently this was combined with swept axial scanning to allow mapping of activity along multiple adjacent dendritic segments (FIG. 4c).

Aspect 1: MROI Scanner Assembly Prototype

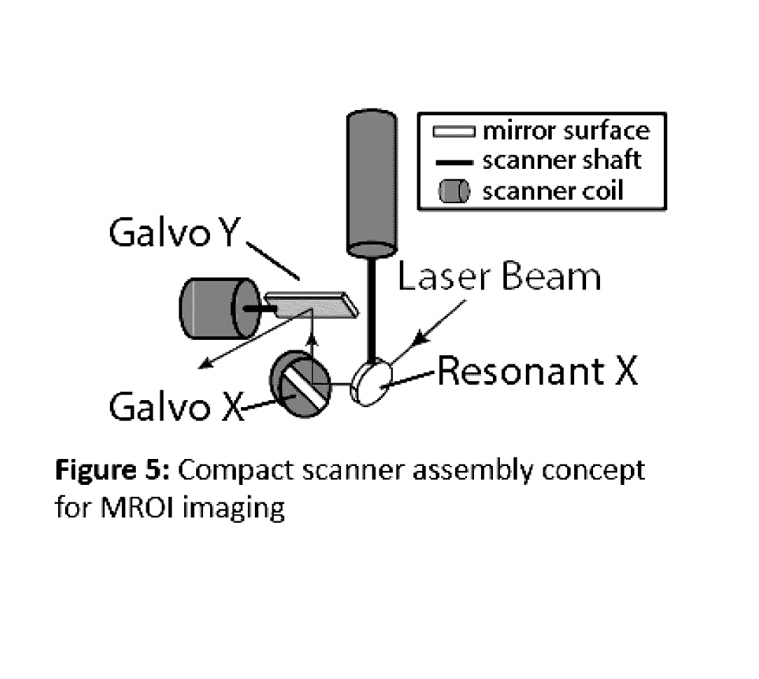

Referring now to FIG. 5, a compact scanner assembly is designed and fabricated to mount and align the scanner surfaces (mirrors) of one resonant and two galvo scanners (FIG. 5).

We have identified the following key design criteria for this:

Scanner étendue (aperture-angle product) of >100 mm-degrees
a. Scan at or near diffraction limit across 1 mm diameter with current objectives

Avoid relay optic couplings to minimize assembly size
a. Integrate readily into existing microscope systems

Minimize inter-scanner distances
a. Minimize pupil shift effect limiting power throughput at large angles

Soundproofed housing (to below 40 dB)
a. Avoid sound distractors of animal behavior and learning experiments

Design and Manufacture

The Nikon CFI 75 16× is a representative recent wide-FOV objective with full entrance aperture and angular range values of 18 mm and 9 degrees, respectively (162 mm-degrees of étendue). Full aperture & angle imaging through this objective achieves diffraction-limited imaging across a reported field of 1.8 mm diameter, but requires the use of additional custom relay optics⁶ (see Risks & Alternatives). We provide the use of a more compact assembly to achieve ease of integration with existing microscope systems, while still achieving >1 mm FOV.

Referring again to FIG. 5, a scanner assembly of 5 mm aperture size and 20 degree angular range (100 mm-degrees) is built from standard components (CRS 8 resonant and 6215H/6 mm galvo scanners; Cambridge Technology). These are selected and ordered such that each successive mirror is larger than the previous mirror: Resonant X (5 mm), Galvo X (6 mm), Galvo Y (6 mm elongated). This, along with maximal close packing, will ensure the full angular range of each scanner is incident on each successive scanner (FIG. 5).

Close packing of the scanner furthermore minimizes “pupil shift” caused by the axial displacement between the scanner surfaces. In 2PLSM systems, scanners are imaged to the objective entrance by telescope optics of magnification M, in order to maximize filling of the objective and thereby the resulting optical resolution²⁵. Axial displacement is also imaged to the objective entrance, causing beam to be shifted on the entrance pupil (i.e. pupil shift) at large scan angles, reducing power throughput. The axial displacement at objective is more strongly magnified by M² (e.g. M=4× to magnify 5 mm scanner to 20 mm objective aperture results in 16× axial displacement magnification). Thus minimizing scanner separation is important.

Despite the pupil-shift effect, virtually all existing 2PLSM systems already use closely spaced RG or GG scanner pairs. Our provided three scanner design adds minimal amount since the thin-shafted resonant scanner is placed very closely to the subsequent X galvo (FIG. 5). Additionally, this configuration allows resonant scanner to extend vertically away from the optical axis. Consequently, the lateral footprint of assembly remains nearly unchanged from a standard GG scanner pair, allowing for ease of integration into existing 2PLSM optical trains.

An initial design of the RGG scanner block is developed using mechanical engineering skills on staff. This design refines the design, adds a soundproofed enclosure, and adds cable connector blocks.

Evaluation

The prototype assemblies are tested using a collimated laser diode (CPS180; Thorlabs), expanded to a 5 mm beam, at the entrance, and a simple photodiode at the output. The GG pair is positioned to central, intermediate, and full deflection angles across the +/−10° range in each dimension. At each 2D angular coordinate, the output beam diameter and relative power is measured, to ensure that a 5 mm diameter and >50% transmission (relative to central angle) is achieved throughout the 20° angular range.

Risks & Alternatives

The current design adds pupil shift beyond current 2PLSM scopes which may lead to significant power dropoff towards edges of the FOV, possibly falling to <50% transmission relative to central angle. A more optimal optical design for the RGG scanner assembly eliminates pupil shift via afocal relay optics between scanners²⁶. Such optics are further optimized, together with the microscope scan optics, to avoid off-axis optical aberrations that otherwise prevent the full utilization of étendue beyond 100 mm-degrees. Optimizing to support >150 mm-degrees maximizes the FOV utilization of both existing (e.g. Nikon CFI 75 16×) and emerging wide-FOV objectives⁶. This design is not a compact upgrade path for existing 2PLSM systems, but is used for new MROI 2PLSM systems.

Aspect 2: MROI Imaging System Prototype

Referring now to FIG. 4, a software prototype is developed for MROI volume imaging using arbitrary RGG scanning hardware (including compact configurations such as in Aspect 1). This software is built on the backbone of Vidrio's ScanImage production hardware and software for resonant raster scanning 2PLSM (FIG. 4a). Using this software prototype, MROI optical recordings in two or more separated cortical volumes may be shown in customer laboratories.

Lateral MROI Imaging

Software will generate & acquire small raster scans at each ROI and then reposition the GG pair to each subsequent ROI with command for shortest transit time possible, computed from a two-parameter model sufficient to describe galvo control systems: acceleration and max velocity¹⁰.

The backbone ScanImage platform uses field-programmable gate array (FPGA) hardware to process high-speed input light data at each resonant scanner period into image lines. For MROI optical recordings, the imaging and non-imaging (inter-ROI transit) times will be discretized into integer sets of the 8 kHz resonant scanner periods, with transit periods being discarded at the FPGA level.

Because the pupil-shift effect and other factors limits power transmission as a function of scan angle, power normalization is implemented whereby power is increased towards the edges of the FOV. This normalization is superimposed atop power normalization for each resonant scanned ROI, to compensate for the variable dwell time at each pixel.

Volume MROI Imaging

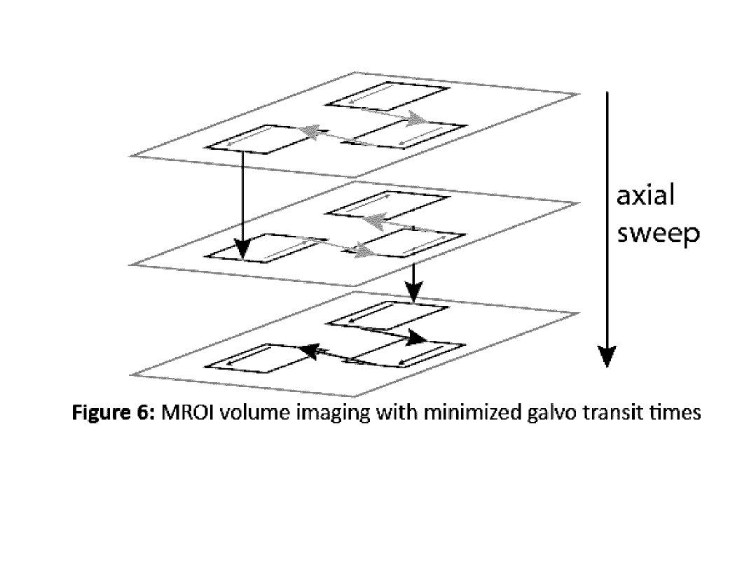

Referring now to FIG. 6, users are provided the ability to select ROIs in two-dimensions from a reference image or image stack recently collected via resonant or galvo raster scanning (latter having a larger angular range). For three-dimensional volume imaging, an axial sweep scan command signal is generated for analog control of focus, e.g. using either a piezoelectric actuator or an electrically tuned lens (Optotune). The axial sweep is synchronized to a user-specified integer number Z of axial planes over a specified range, each containing a set of 2 or more selected ROIs, with an ordering & image directionality to optimize overall galvo transit time (FIG. 6). Volume imaging rates are F/(Z+1), where F is the 2D MROI frame rate, with one frame period discarded to allow for the axial scanner flyback. For instance, imaging of 3 ROIs of 4× zoom through 8 axial planes is achieved at ˜8 Hz with 64×64 pixel imaging (Table 1).

Evaluation

An MROI imaging prototype system is tested. MROI volume imaging of neural ensemble activity in 2 or more distinct cortical columns is shown at >4 Hz and >100 neurons per ensemble. For 1 mm diameter FOV (Aspect 1), specifying ROI zoom factors of 4×(250 μm diameter) and a set of 8 planes (e.g. 30 μm spacing) would be expected to contain >100 cells in even sparsely labeled subjects, with expected rates of ˜8 Hz and ˜4 Hz for 3 and 4 ROIs, respectively.

For each MROI volume imaging dataset, a comparison acquisition is obtained using conventional raster volume imaging. The MROI advantage is determined by computing the neuronal volume imaging figure-of-merit M:

M=Tᵥₒₗ×Σᵢ^{N}^{_{ROI}} SNRᵢ

where Tᵥₒₗ is the imaging period for the full volume, SNRᵢ is the signal-to-noise ratio assessed at each ROI, and N_{ROI} is the number of ROIs selected for MRoI imaging. For an ROI zoom factor of 4×, SNR increases of up to 4× per ROI and aggregate frame speed increases up to 2× would nominally achieve M=16 regardless of scenario chosen (Table 1), with real values of M>5 after accounting for inter-ROI transit times and possible sublinear scaling of SNR with pixel integration time.

Risks and Alternatives

A stepped axial scanning mode was considered using a fast stepping electrically tuned lens²⁷. This would avoid the tilting of the image volume that occurs with the swept axial scan provided. But given best current specifications (Optotune; 7 ms axial step time), this approach was determined to discard too much time for each set of axial planes.

Increasing dwell time at each pixel does not linearly augment the SNR owing to higher order photophysical effects in 2PLSM²⁸. This limits the MROI figure-of-merit M achieved, but substantial gains in speed and/or signal quality are nonetheless achieved.

### REFERENCES CITED

1. MOUNTCASTLE V B. Modality and topographic properties of single neurons of cat's somatic sensory cortex. J Neurophysiol. 1957; 20(4):408-434. http://www.ncbi.nlm.nih.gov/pubmed/13439410. Accessed Jan. 27, 2015.

2. Mountcastle V B. The columnar organization of the neocortex. Brain. 1997; 120 (Pt 4:701-722. http://www.ncbi.nlm.nih.gov/pubmed/9153131. Accessed Feb. 26, 2015.

3. Hubel D H, Wiesel T N, Stryker M P. Orientation columns in macaque monkey visual cortex demonstrated by the 2-deoxyglucose autoradiographic technique. Nature. 1977; 269(5626):328-330. http://www.ncbi.nlm.nih.gov/pubmed/409953. Accessed Mar. 27, 2015.

4. Leise E M. Modular construction of nervous systems: a basic principle of design for invertebrates and vertebrates. Brain Res Brain Res Rev. 15(1):1-23. http://www.ncbi.nlm.nih.gov/pubmed/2194614. Accessed Mar. 27, 2015.

5. Li N, Chen T-W, Guo Z V., Gerfen C R, Svoboda K. A motor cortex circuit for motor planning and movement. Nature. 2015; 519(7541):51-56. doi:10.1038/nature14178.

6. Stirman J N, Smith I T, Kudenov M W, Smith S L. Wide Field-of-View, Twin-Region Two-Photon Imaging across Extended Cortical Networks. Cold Spring Harbor Labs Journals; 2014. doi:10.1101/011320.

7. Chen T-W, Wardill T J, Sun Y, et al. Ultrasensitive fluorescent proteins for imaging neuronal activity. Nature. 2013; 499(7458):295-300. doi:10.1038/nature12354.

8. Fan G Y, Fujisaki H, Miyawaki A, Tsay R K, Tsien R Y, Ellisman M H. Video-rate scanning two-photon excitation fluorescence microscopy and ratio imaging with cameleons. Biophys J. 1999; 76(5):2412-2420. doi:10.1016/S0006-3495(99)77396-0.

9. Keller G B, Bonhoeffer T, Hithener M. Sensorimotor mismatch signals in primary visual cortex of the behaving mouse. Neuron. 2012; 74(5):809-815. doi:10.1016/j.neuron.2012.03.040.

10. Lillis K P, Eng A, White J A, Mertz J. Two-photon imaging of spatially extended neuronal network dynamics with high temporal resolution. J Neurosci Methods. 2008; 172(2):178-184. doi:10.1016/j.jneumeth.2008.04.024.

11. Sadovsky A J, Kruskal P B, Kimmel J M, Ostmeyer J, Neubauer F B, MacLean J N. Heuristically optimal path scanning for high-speed multiphoton circuit imaging. J Neurophysiol. 2011; 106(3):1591-1598. doi:10.1152/jn.00334.2011.

12. Cotton R J, Froudarakis E, Storer P, Saggau P, Tolias A S. Three-dimensional mapping of microcircuit correlation structure. Front Neural Circuits. 2013; 7:151. doi:10.3389/fncir.2013.00151.

13. Lecoq J, Savall J, Vu\check{c}inić D, et al. Visualizing mammalian brain area interactions by dual-axis two-photon calcium imaging. Nat Neurosci. 2014; 17(12):1825-1829. doi:10.1038/nn.3867.

14. Froudarakis E, Berens P, Ecker A S, et al. Population code in mouse V1 facilitates readout of natural scenes through increased sparseness. Nat Neurosci. 2014; 17(6):851-857. doi:10.1038/nn.3707.

15. Iyer V, Hoogland T M, Saggau P. Fast functional imaging of single neurons using random-access multiphoton (RAMP) microscopy. J Neurophysiol. 2006; 95(1):535-545. doi:10.1152/jn.00865.2005.

16. Duemani Reddy G, Kelleher K, Fink R, Saggau P. Three-dimensional random access multiphoton microscopy for functional imaging of neuronal activity. Nat Neurosci. 2008; 11(6):713-720. doi:10.1038/nn.2116.

17. Katona G, Szalay G, Maák P, et al. Fast two-photon in vivo imaging with three-dimensional random-access scanning in large tissue volumes. Nat Methods. 2012; 9(2):201-208. doi:10.1038/nmeth.1851.

18. Valmianski I, Shih A Y, Driscoll J D, Matthews D W, Freund Y, Kleinfeld D. Automatic identification of fluorescently labeled brain cells for rapid functional imaging. J Neurophysiol. 2010; 104(3):1803-1811. doi:10.1152/jn.00484.2010.

19. Smith W. Modern Optical Engineering. 4th ed. New York, N.Y.: McGraw-Hill; 2007.

20. Prakash R, Yizhar O, Grewe B, et al. Two-photon optogenetic toolbox for fast inhibition, excitation and bistable modulation. Nat Methods. 2012; 9(12):1171-1179. doi:10.1038/nmeth.2215.

21. Rickgauer J P, Deisseroth K, Tank D W. Simultaneous cellular-resolution optical perturbation and imaging of place cell firing fields. Nat Neurosci. 2014; 17(12):1816-1824. doi:10.1038/nn.3866.

22. Packer A M, Peterka D S, Hirtz J J, Prakash R, Deisseroth K, Yuste R. Two-photon optogenetics of dendritic spines and neural circuits. Nat Methods. 2012; 9(12):1202-1205. doi:10.1038/nmeth.2249.

23. Rickgauer J P, Tank D W. Two-photon excitation of channelrhodopsin-2 at saturation. Proc Natl Acad Sci USA. 2009; 106(35):15025-15030. doi:10.1073/pnas.0907084106.

24. Williams S C P, Deisseroth K. Optogenetics. Proc Natl Acad Sci USA. 2013; 110(41):16287. doi:10.1073/pnas.1317033110.

25. Zipfel W R, Williams R M, Webb W W. Nonlinear magic: multiphoton microscopy in the biosciences. Nat Biotechnol. 2003; 21(11):1369-1377. doi:10.1038/nbt899.

26. Tsai P S, Kleinfeld D. In vivo two-photon laser scanning microscopy with concurrent plasma-mediated ablation. In: Frostig R, ed. Methods for In Vivo Optical Imaging. CRC Press; 2009: 59-115.

27. Sheffield M E J, Dombeck D A. Calcium transient prevalence across the dendritic arbour predicts place field properties. Nature. 2014. doi:10.1038/nature13871.

28. Patterson G H, Piston D W. Photobleaching in two-photon excitation microscopy. Biophys J. 2000; 78(4):2159-2162. doi:10.1016/S0006-3495(00)76762-2.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

Notes

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine-implemented or computer implemented at least in part. Some examples can include a tangible computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed as the invention:

1. An apparatus for targeting a sample of intact cellular tissue, the apparatus comprising:
a source of beamed electromagnetic energy producing a beam;
a compact scanner assembly for multi-region of interest (MROI) angular scanning of the beam; and
optics for imaging the angular scanned beam from scanner assembly to a microscope objective lens, wherein the microscope objective lens converts the angular scanned beam to a focused spatial-focusing scanned beam onto intact cellular tissue.

2. An apparatus for imaging a sample of intact cellular tissue, the apparatus comprising:
a source of beamed electromagnetic energy producing a beam;
a compact scanner assembly for multi-region of interest (MROI) angular scanning of the beam;
optics for imaging the angular scanned beam from scanner assembly to a microscope objective lens, wherein the microscope objective lens converts the angular scanned beam to a focused spatial-focusing scanned beam onto intact cellular tissue; and
a detector to detect the resulting radiation signal from the sample region.

3. A compact scanner assembly for multi-region of interest (MROI) imaging of intact cellular tissue, comprising a set of three electromagnetically actuated scanning mirrors, the first scanning mirror comprising a resonant scanner (R) driven at its resonant frequency, the second scanning mirror comprising a galvanometer scanner (G1) having a mirror (m1), the third scanning mirror comprising a galvanometer scanner (G2) having a mirror (m2), wherein the galvanometer scanner (G1) and the galvanometer scanner (G2) are driven by a lower bandwidth control signal specifying an angle for mirror (m1) and for mirror (m2), wherein the scanners are arranged sequentially as the resonant scanner (R) followed by the galvanometer scanner (G1) and the galvanometer scanner (G2), wherein the set of three scanning mirrors are within a single scanner assembly (RGG).

4. The compact scanner assembly of claim 3, wherein the set of three scanning mirrors are oriented within the compact scanner assembly to produce a two-dimensional (X & Y) angular scan at an assembly output, wherein the resonant scanner (R) and the galvanometer scanner (G1) are oriented to produce scanning in the same (X) angular direction.

5. The compact scanner assembly of claim 3, wherein the compact scanner assembly for MROI combines raster scanning with random-access scanning to target multiple geometric ROIs for imaging.

6. The compact scanner assembly of claim 5, wherein the geometric ROIs comprise a linear ROI, a planar ROI, or a three-dimensional ROI.

7. The compact scanner assembly of claim 6, wherein the geometric ROIs comprise a rectangular ROI, a line ROI, a curved line ROI, a circular ROI, an elliptical ROI, a multi-sided ROI, a polygon ROI, an irregular ROI, a cubic ROI, a prismatic ROI, a cylindrical ROI, or a spherical ROI.

8. A method of imaging a sample, comprising:
scanning a beam of electromagnetic energy over an intact tissue sample;
using a compact scanner assembly for multi-region of interest (MROI) imaging of intact cellular tissue disposed to receive the beam; and
detecting the resulting radiation signal from the sample region.

9. A method for providing multi region cellular resolution imaging, comprising the steps: providing a device as in claim 1-3, using the device of claim 1-3 to obtain MROI images.

10. A method of targeting a sample of cellular tissue, comprising:
directing a beam of electromagnetic energy at an intact tissue sample; and
using a compact scanner assembly for multi-region of interest (MROI) targeting of intact cellular tissue disposed to receive the beam.

11. A method for providing multi region cellular resolution targeting, comprising the steps: providing a device as in claim 1-3, using the device of claim 1-3 to target cellular tissue.

12. The method as in claim 11 or 12, wherein the cellular tissue is a synapse.

13. The method as in claim 11 or 12, further comprising the step of ablating of the targeted cells.

14. The method as in claim 11 or 12, further comprising the step of optogenically stimulating the targeted cells.

15. The method as in claim 11 or 12, further comprising the step of photo-stimulating the targeted cells.

16. The method as in claims 10-15, wherein the compact scanner assembly for MROI combines raster scanning with random-access scanning to target multiple geometric ROIs for targeting wherein the geometric ROIs comprise a linear ROI, a planar ROI, or a three-dimensional ROI.

17. The method as in claim 16, wherein the geometric ROIs comprise a rectangular ROI, a line ROI, a curved line ROI, a circular ROI, an elliptical ROI, a multi-sided ROI, a polygon ROI, an irregular ROI, a cubic ROI, a prismatic ROI, a cylindrical ROI, or a spherical ROI.
